(19) United States
(12) Patent Application Publication
(10) Pub. No.: US 2026/0364354 A1
(43) Pub. Date: Nov. 19, 2026

(54) IMPLANTABLE STIMULATION SYSTEMS AND METHODS FOR BLOOD PRESSURE CONTROL

(71) Applicant: The Alfred E. Mann Foundation for Scientific Research, Valencia, CA (US)

(72) Inventors: Brian MECH, Buffalo, MN (US); Lee STYLOS, Stillwater, MN (US)

(21) Appl. No.: 19/578,548

(22) Filed: Mar. 25, 2026

Related U.S. Application Data
(60) Provisional application No. 63/808,492, filed on May 19, 2025.

(51) Int. Cl. A61N 1/36; A61N 1/05

(52) U.S. Cl. CPC A61N 1/36117; A61N 1/36139; A61N 1/0556

(57) ABSTRACT

The present disclosure generally relates to systems and methods for reducing blood pressure in a human subject, while reducing power consumption in the system and reducing or eliminating unwanted side effects experienced by the subject.

### CROSS-REFERENCE TO RELATED APPLICATION

The present application claims the benefit of U.S. Provisional Application No. 63/808,492, entitled “MEANS OF STIMULATION TO REDUCE BLOOD PRESSURE,” filed May 19, 2025, which is assigned to the assignee hereof and is expressly incorporated herein by reference in its entirety.

### TECHNICAL FIELD

This disclosure relates generally to implantable stimulation systems and methods for controlling blood pressure. More particularly, the disclosure relates to electrical stimulation of baroreceptors and/or neural pathways associated with baroreceptor signaling, including the carotid sinus nerve (CSN), to modulate blood pressure.

### BACKGROUND

Hypertension, or high blood pressure (BP), is a significant risk factor for cardiovascular diseases, including heart attack and stroke. While conventional treatments such as lifestyle modifications and pharmacological interventions are effective for many individuals, a subset of patients experience uncontrolled hypertension. As used herein, “uncontrolled hypertension” refers to hypertension that remains above a desired target level despite one or more antihypertensive interventions, and includes, without limitation, drug-resistant hypertension (RHT) as well as hypertension that is uncontrolled due to poor, inconsistent, impractical, contraindicated, or otherwise not possible medication use or adherence (e.g., intolerance, side effects, access limitations, or noncompliance). This challenge has driven the search for alternative treatment approaches.

Interest in neural stimulation as a potential method for lowering blood pressure has grown in recent years. Prior clinical and investigational efforts have demonstrated that stimulating baroreceptors in the carotid sinus can effectively reduce BP in human patients, at least in the short term. However, prior systems have faced challenges related to safety, tolerability, procedural complexity, and/or sustained long-term efficacy, including stimulation levels that may cause undesired side effects in some patients.

Presently, there are no implantable stimulation devices that are suitable for chronic management of BP, including for patients with uncontrolled hypertension. Although prior approaches demonstrated the efficacy of such treatments for lowering BP acutely, numerous questions remain regarding how such therapy can be implemented safely and effectively for long-term use. Specifically, it is unclear how much stimulation is required, when it should be applied, and how frequently it must be delivered to sustain a lowered BP (or to further reduce BP over time).

### BRIEF SUMMARY

The following presents a concise summary of one or more aspects to provide a basic understanding of such aspects. This summary is not an extensive overview of all contemplated aspects and is intended to neither identify key nor critical elements of all aspects nor delineate the scope of any or all aspects. Its sole purpose is to present some concepts of one or more aspects in a simplified form as a prelude to the more detailed description that is presented later.

Aspects of the disclosure are directed to an implantable apparatus configured for reducing blood pressure in a human subject. In some examples, the implantable apparatus includes one or more memories, individually or in combination, having instructions. In some examples, the implantable apparatus includes one or more processors, individually or in combination, configured to execute the instructions. In some examples, the one or more processors, individually or in combination, are configured to detect a metric associated with a physiological event of the human subject, the metric satisfying a threshold condition. In some examples, the one or more processors, individually or in combination, are configured to adjust one or more parameters associated with a carotid sinus nerve (CSN) or baroreceptor stimulation protocol. In some examples, the one or more processors, individually or in combination, are configured to apply stimulation to the CSN according to the adjusted one or more parameters.

In some aspects, the metric comprises at least one of a heart rate, a respiration rate, an activity level, a time of day, or a blood pressure.

In some aspects, the one or more processors, individually or in combination, are further configured to: receive, from an external device via a wired or wireless interface, an indication of the threshold condition and an indication of the metric associated with the threshold condition.

In some aspects, the one or more parameters associated with the CSN stimulation protocol comprises: a periodicity or frequency associated with the application of stimulation, a timing associated with the application of stimulation, an intensity of the stimulation, or a duration of the application of stimulation.

In some aspects, the physiological event is a first QRS complex, wherein the stimulation applied to the CSN is a first stimulation, and wherein first stimulation to the CSN is applied X milliseconds after detection of the first QRS complex.

In some aspects, the one or more processors, individually or in combination, are further configured to: detect a second QRS complex occurring after the first QRS complex and after the first stimulation is applied; and refrain from applying a second stimulation to the CSN until after detection of an Nth QRS complex.

In some aspects, stimulation to the CSN is applied X milliseconds after every Nth detected QRS complex.

Aspects of the disclosure are directed to a method for reducing blood pressure in a human subject. In some examples, the method includes detecting a metric associated with a physiological event of the human subject, the metric satisfying a threshold condition. In some examples, the method includes adjusting one or more parameters associated with a carotid sinus nerve (CSN) stimulation protocol. In some examples, the method includes applying stimulation to the CSN according to the adjusted one or more parameters.

In some aspects of the methods described herein, the metric comprises at least one of a heart rate, a respiration rate, an activity level, a time of day, or a blood pressure.

In some aspects, such methods further comprise a step of receiving, from an external device via a wired or wireless interface, an indication of the threshold condition and an indication of the metric associated with the threshold condition.

In some aspects of the methods described herein, the one or more parameters associated with the CSN stimulation protocol comprises: a periodicity or frequency associated with the application of stimulation, a timing associated with the application of stimulation, an intensity of the stimulation, or a duration of the application of stimulation.

In some aspects of the methods described herein, the physiological event is a first QRS complex, wherein the stimulation applied to the CSN is a first stimulation, and wherein first stimulation to the CSN is applied X milliseconds after detection of the first QRS complex.

In some aspects, such methods further comprise the steps: detecting a second QRS complex occurring after the first QRS complex and after the first stimulation is applied; and refraining from applying a second stimulation to the CSN until after detection of an Nth QRS complex.

In some aspects of the methods described herein, stimulation to the CSN is applied X milliseconds after every Nth detected QRS complex.

Aspects of the disclosure are directed to a non-transitory, computer-readable medium comprising computer executable code. The code when executed by one or more processors causes the one or more processors to, individually or in combination, perform operations configured to reduce blood pressure in a human subject. In some examples, the operations include detecting a metric associated with a physiological event of the human subject, the metric satisfying a threshold condition. In some examples, the operations include adjusting one or more parameters associated with a carotid sinus nerve (CSN) stimulation protocol. In some examples, the operations include applying stimulation to the CSN according to the adjusted one or more parameters.

Aspects of the disclosure are directed to an apparatus configured for reducing blood pressure in a human subject. In some examples, the apparatus includes means for detecting a metric associated with a physiological event of the human subject, the metric satisfying a threshold condition. In some examples, the apparatus includes means for adjusting one or more parameters associated with a carotid sinus nerve (CSN) stimulation protocol. In some examples, the apparatus includes means for applying stimulation to the CSN according to the adjusted one or more parameters.

### BRIEF DESCRIPTION OF THE DRAWINGS

FIG. 1 is an anatomical diagram illustrating an example of a carotid artery in a human neck, including relative positions of the carotid sinus, the carotid sinus nerve (CSN), as well as an arterial cuff and a corresponding implantable pulse generator (IPG).

FIG. 2 is a flow chart illustrating an example stimulation protocol configured to stimulate the CSN in response to less than every detected QRS complex.

FIG. 3 is a flow chart illustrating an example process for adjusting a stimulation protocol configured to stimulate the CSN.

FIG. 4 is a flow diagram illustrating an example method for reducing blood pressure in a human subject.

FIG. 5 is a diagram illustrating waveforms of arterial blood pressure, ECG, and stimulation amplitude. In this case, a burst of stimulation is delivered during the pulse arrival time (“PAT”) every N cardiac cycles. Here, N=2. Alternative values for N, and other stimulation patterns are also envisioned and described further herein.

### DETAILED DESCRIPTION

The following presents a description of one or more aspects of the present disclosure to provide a basic understanding. Any or all of the discussed aspects/features could be interchangeably applied between examples, and, unless otherwise stated, no single aspect or feature is essential to achieve the technical effects/solutions discussed.

Chronic hypertension remains a significant clinical problem, particularly for patients whose blood pressure does not adequately respond to medication, or have difficulty complying with medication. Although baroreceptor stimulation has been shown to acutely lower blood pressure in humans, the field has not solved how to translate that acute effect into a durable, safe, and power efficient long-term therapy. Prior efforts—most notably devices targeting the carotid sinus—demonstrated short term blood pressure reductions but encountered significant safety and tolerability challenges, including off target autonomic effects, as well as trial design and regulatory hurdles.

Some issues relating to safety and tolerability challenges relate to how much stimulation to deliver, when to deliver it, and how frequently to deliver it to sustain blood pressure reductions without provoking undesirable side effects or exhausting device power budgets. Conventional approaches default toward continuous stimulation to maintain efficacy. That “always on” posture drives up energy consumption, shortens primary cell battery longevity, and, in the case of a rechargeable battery, increases recharge burden—problems that are particularly acute for implanted pulse generators.

Moreover, physiologic complexities at the nerve targets present additional issues. The carotid sinus nerve carries both baroreceptor input and chemoreceptor input from the carotid body; stimulating the former can lower blood pressure, but indiscriminate stimulation risks interfering with chemoreflex signaling and can affect heart rate or respiration. For example, the carotid body, upstream near the bifurcation of the carotid artery, also contains several chemoreceptors that typically monitor oxygen, carbon dioxide, and pH levels and send signals to the brain, via the carotid sinus nerve (CSN), that can change respiration and cardiovascular function. Thus, when one stimulates the CSN, it is possible to interfere with/alter the transmission of these chemoreceptor signals. This could induce undesired changes in cardiovascular function (outside of blood pressure) such as the heart rate and/or respiration rate. Similarly, the vagus nerve (VN), which can be accessed very close to the CSN, contains afferent and efferent nerve fibers that impact many aspects of human control system (e.g., heart rate, digestion, respiration, mood, immune response, and others).

Finally, there is no existing framework for synchronizing stimulation with the patient's intrinsic physiology to maximize baroreflex activation while minimizing unwanted collateral effects. For example, the time difference between the electrical pulse from the heart (e.g., represented by the QRS complex on an electrocardiogram (EKG)) and the arrival of the mechanical pulse at the carotid artery can typically be anywhere between 100 milliseconds (ms) and 300 ms. This is also known as the pulse arrival time (PAT). The delay is caused by the time it takes for the electrical signal to produce a heart contraction and for the resulting pressure wave to travel from the heart through the aorta to the carotid artery. A stimulating pulse at the CSN would ideally occur within the PAT (e.g., after the electrical pulse from the heart and prior to the mechanical pulse at the carotid artery). However, without such synchronization of the system with the PAT, therapeutic effect of the stimulation may be reduced or lost entirely.

Accordingly, aspects of the disclosure are directed to systems and methods for a physiologically synchronized approach to baroreceptor and related nerve stimulation configured to materially reduce power consumption, extend device longevity, and improve safety by minimizing off target autonomic effects. It may also be the case that therapeutic effectiveness is increased or maintained versus an always-on approach to stimulation.

In certain aspects, the system may monitor the electrical (e.g., QRS) activity of a subject's heart using any suitable method. The system may then deliver a stimulation to the CSN (a particular baroreceptor that can be accessed in the neck of a patient, at a pre-configured time occurring after the electrical activity and within the PAT. Timing the stimulation therapy around this event allows the system to work with the body's natural rhythm at the point just before blood is pumped out, which is when stimulation is most likely to engage the baroreflex (e.g., the body's blood-pressure-lowering reflex) and least likely to accidentally trigger other nerves that control heart rate or breathing. In other words, better timing yields of the CSN stimulation yields more blood-pressure benefit with fewer side effects.

Moreover, the system may refrain from stimulating the CSN at every heartbeat. For example, the system may be programmed to stimulate at every Nth beat (e.g., every 2nd, 3rd, or 4th beat). By treating every Nth beat, power consumption can be reduced, thereby extending battery life and reducing recharging frequency and/or battery replacement, without sacrificing the beneficial therapeutic effects on the subject's blood pressure. In some examples, different values of N may be used on a particular subject to determine an optimal N value that results in keeping blood pressure at an acceptable level while reducing power consumption.

This disclosure primarily discusses baroreceptor stimulation with reference to three exemplary targets in the neck: the carotid sinus, the carotid sinus nerve, and the vagus nerve, although baroreceptor targets could be stimulated elsewhere in the body as well.

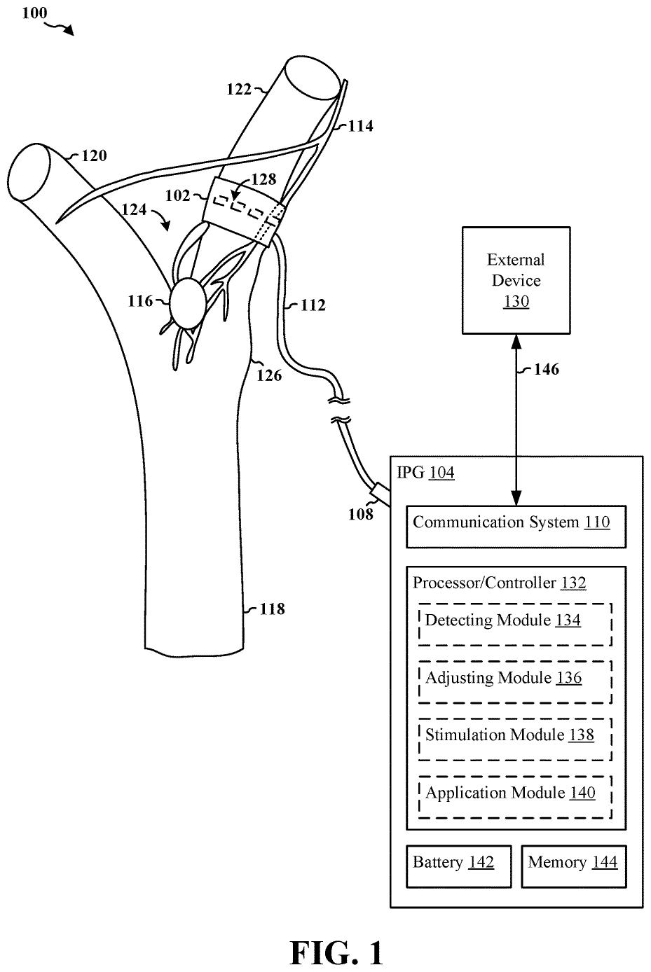

FIG. 1 is an anatomical diagram illustrating an example 100 of a carotid artery in a human neck, including relative positions of the carotid sinus 126, the carotid sinus nerve (CSN) 114, as well as an arterial cuff 102 and a corresponding implantable pulse generator (IPG) 104, according to aspects of the disclosure. As shown in FIG. 1, the carotid body 116, a small, ovoid-shaped (e.g., sometimes described as approximately the size of a grain of rice), and highly vascularized organ is situated in or near a carotid bifurcation 124, where the common carotid artery 118 branches into an internal carotid artery (IC) 122 and external carotid artery (EC) 120.

The CSN 114 is positioned downstream (i.e. closer to the head) of the carotid sinus 126 and is typically attached to the IC 122. The CSN 114 exists on both sides of the human body (bilaterally) and can lower systemic blood pressure when stimulated, even on just one side. The carotid body 116, located upstream near the bifurcation 124 of the carotid artery 118, houses chemoreceptors that monitor oxygen, carbon dioxide, and pH levels. These chemoreceptors send signals to the brain via the CSN 114 to regulate respiration and cardiovascular function. However, because stimulating the CSN 114 can interfere with these signals, without a methodical approach, stimulation of the CSN to control BP may inadvertently cause unwanted changes in cardiovascular function, including changes in heart rate and/or respiration rate.

A similar situation applies to the vagus nerve (VN), which may be located near the CSN 114 and is also present bilaterally. The VN contains both afferent and efferent nerve fibers that influence numerous physiological functions, including heart rate, digestion, respiration, mood, and immune response. Unless stimulation is precisely directed at the baroreceptors within the VN, those originating from the aortic arch, there is a risk of inducing unintended side effects.

The arterial cuff 102 may include one or more electrodes 128 embedded or otherwise attached to the arterial cuff 102. The arterial cuff 102 may be a ring composed of silicone or any other medically suitable material, and the electrodes 128 may be composed any suitable electrically conductive material or combination of materials (e.g., aluminum, nickel, platinum, iridum, or alloys thereof, etc.). The arterial cuff 102 and the electrodes 128 may configured such that the electrodes 128 make direct or indirect contact with the CSN 114. For example, the arterial cuff 102 may be attached to the IC 122 during a surgical procedure, and the arterial cuff 102 may be oriented such that one or more of the electrodes 128 are located on top of or over a portion of the CSN 114. In such a configuration, an electrode to which an electrical pulse is applied may pass that pulse to the CSN 114 to stimulate the CSN 114. In another example (not pictured), the arterial cuff could be placed around the biological tissue between the two branches of the carotid artery, such that it contains that CSN within that tissue bundle.

Each of the one or more electrodes 128 may be configured to receive an electrical charge via an attached wire encased within a lead cable 112 that attaches the arterial cuff 102 to the IPG 104. For example, if the arterial cuff 102 has four electrodes, then the lead cable 112 may include four wires that run between the arterial cuff 102 and the IPG 104. In this manner, an electrical pulse may be applied to each electrode individually, independent of other electrodes. Thus, one or more of the electrodes 128 may be configured to deliver an electrical pulse to the CSN 114. This stimulation may be timed to occur after a configured time delay following a detected QRS complex.

The IPG 104 may include a case or housing configured to enclose a battery 142, a communication system 110, one or more processors/controllers 132, one or more memories 144 (e.g., configured to store data, sensor information, and instructions for executing functions associate with the modules described herein), and an interface 108 configured to couple the lead wire and each of the electrodes 128 to the IPG 104. The IPG could also have one or more electrodes on its case that can serve as return electrodes for stimulation done at the cuff.

In certain aspects, the communication system 110 may include hardware and software configured to enable wired (e.g., universal serial bus (USB), peripheral component interconnect express (PCIe)) and/or wireless (e.g., Bluetooth, low-energy Bluetooth, RF, MedRadio, ZigBee, Wi-Fi, etc.) communication 146 between the IPG 104 and an external device 130 (e.g., medical devices and equipment, personal electronic devices, smart phone, laptop, personal digital assistant (PDA), tablet, wearable device, etc.). The wired and/or wireless interfaces may be configured to allow the IPG 104 to communicate information and commands between the IPG 104 and the external device 130. In some examples, the information and commands may allow a user or administrator to modify or select programs within the IPG 104, thereby modifying the operating configuration of the IPG 104.

The IPG 104 may include one or more processors/controllers 132 that, individually or in combination, are configured to perform certain functions. These functions are illustrated as individual modules that each have a hardware and software component configured to perform the respective functions. These modules include a detecting module 134, an adjusting module 136, a stimulation module 138, and an application module 140.

The detecting module 134 may be configured to detect or sense physiological events and status of a human subject within which the IPG 104 is implanted. As such, the detecting module 134 may include one or more sensors that are contained within the housing of the IPG 104. Alternatively, or in addition, the sensors may be affixed to the exterior of the outer case or housing, or on the lead body, or in/on the cuff, or positioned at a remote site within the body and coupled to the IPG 104 with a connecting lead (e.g., lead cable 112), or wirelessly. Such sensors may include one or more electrodes within the arterial cuff 102, or sensors coupled to the external device 130 and coupled to the IPG 104 via a wired or wireless connection. The detecting module 134 may be configured to detect, via the one or more sensors, physiological events such as respiration rate (e.g., motion or ribcage movement), heart rate, QRS complex, R-R intervals, blood pressure, time of day, and/or activity level of the human subject. Suitable sensors include, but are not limited to, inertial sensors, bioimpedance sensors, pressure sensors, gyroscopes, ECG/EKG electrodes, temperature sensors, GPS sensors, and combinations thereof.

The adjusting module 136 may be configured to adjust stimulation parameters (e.g., timing, frequency, and/or intensity) of a stimulation protocol associated with the electrodes 128 and controlled by the stimulation module 138. In some examples, the adjusting module 136 may be configured to adjust the stimulation parameters based on physiological events and/or status of the human subject detected by the detecting module, as described in more detail below.

The stimulation module 138 may be configured to control electrical stimulation of the one or more electrodes 128 in the arterial cuff 102. In some examples, the stimulation module 138 includes an electrical pulse generator configured to energize an electrode via a corresponding wire independent of one or more other electrodes.

The application module 140 may be configured to communicate information and commands with a corresponding application on the external device 130. For example, the application module 140 may receive an indication of a threshold condition (explained in more detail below) configured to set a triggering threshold that causes the IPG 104 to perform an action (e.g., stimulate an electrode) or refrain from performing an action. Accordingly, a medical practitioner or the human subject that receives the IPG 104 may modify the functionality of the IPG 104 from an external device 130 via the application module 140. In some examples, the medical practitioner or the human subject may modify an event being monitored and detected by the detecting module 134 (e.g., by setting the system to monitor an alternative event, or a plurality of events). Such events may include: QRS complex (e.g., each heart beat waveform), a heart rate, a respiration rate, an activity level, a time of day, and/or a blood pressure. The medical practitioner or the human subject may modify the timing of stimulation, including: frequency (e.g., periodicity of stimulation), timing of the stimulation (e.g., duration of time between detection of a triggering event and application of the stimulation), and intensity of the stimulation (e.g., level of voltage/current applied to the electrode(s) during stimulation, duration of time that voltage/current pulse is applied to the electrode(s), a quantity of pulses applied to the electrode(s), etc.).

In certain aspects, the memory 144 stores data gathered by the detecting module 134, and/or data and information provided by the external device 130. The memory 144 may also store programming instructions and stimulation parameters/protocol. The stimulation module 138 may use the stored data and information to control stimulation of one or more electrodes 128. For example, the stimulation protocol may instruct the stimulation module 138 to apply a particular current (e.g., a current within the range of 0.1 to 10 mA, or 1.0 to 6.0 mA, including any subranges therebetween) or voltage value to a first electrode 200 ms after an Nth QRS interval is detected. Here, the stimulation module 138 may increment a counter with each QRS interval detected by the detecting module 134. When the counter reaches N (e.g., where N is a non-zero whole number, e.g., 1, 2, 3, 4, 5, 6, 7, 8, 9, or 10), the stimulation module 138 may delay stimulating the first electrode by 200 ms after the Nth QRS interval is detected. The counter may then be reset after reaching N and stimulating the first electrode. To be clear, this is a non-limiting example, In further aspects, the current or voltage may be applied to a single electrode or a plurality of electrodes, and the delay may comprise 100, 150, 200, 250, 300, 350, 400, 450 or 500 ms, or a delay comprising any integer value or a subrange defined by any pair of integer values, therebetween.

The adjusting module 136 may be configured to receive and analyze data detected by the detecting module 134 and adjust the stimulation parameters/protocol based on the data. For example, the adjusting module may be configured to adjust the stimulation protocol such that the stimulation module 138 refrains from energizing any of the electrodes 128 if the data collected by the detecting module 134 indicates that the human subject is sleeping or exercising. In one example, the detecting module 134 may determine that the current time of day is within a time window (e.g., 9 PM to 6 AM) within which the human subject typically sleeps. Alternatively, or in combination, the detecting module 134 may determine that the respiratory rate of the human subject satisfies a threshold condition indicative of sleep or exercise, and may thus adjust the stimulation protocol such that the stimulation module 138 refrains from energizing any of the electrodes 128 while the respiratory rate satisfies the threshold condition. As described in more detail below, data detected by the detecting module 134 may trigger adjustment of other stimulation parameters and protocol.

### Examples of Stimulation Protocols Triggered by a QRS Complex

Aspects of the disclosure are directed to stimulation protocols that are triggered by a detected QRS complex. As described here, a stimulation protocol generally relates to a procedure via which the IPG 104 applies a voltage across one or more electrodes 128 that are positioned over the CSN 114 and located on the arterial cuff 102. As discussed above, such a stimulation has been shown to reduce blood pressure. However, it should be noted that the stimulation protocol may not be triggered to stimulate an electrode in response to each detected QRS complex. Limiting the stimulation to less than every QRS complex increases the power efficiency of the IPG 104 by improving battery 142 life, while also eliminating or reducing undesirable side effects of the stimulation.

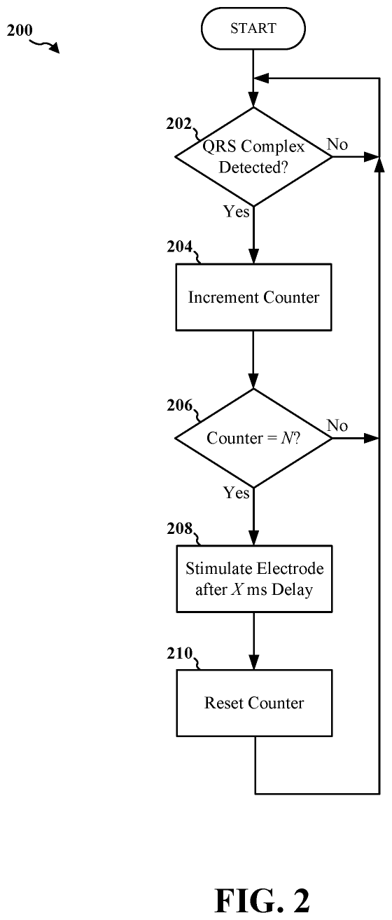

FIG. 2 is a flow chart illustrating an example stimulation protocol 200 configured to stimulate the CSN 114 upon every detected QRS complex (e.g., where N=1), or potentially less than every detected QRS complex (e.g., where N=2, 3, 4, etc.). At a first decision 202, the stimulation protocol 200 determines whether a QRS complex event (e.g., electrical pulse generated by a sinoatrial node of the heart) has been detected. For example, the detecting module 134 may detect the QRS event. If such an event is detected, the stimulation protocol 200 may proceed to a first process 204, wherein the IPG 104 increments a counter in response to the detected QRS complex. Here, the IPG may refrain from applying a voltage to any electrode until the counter satisfies a threshold condition. In some examples, the human subject with the IPG 104 or a medical practitioner may use an external device 130 to set (e.g., via the application module 140) a threshold counter value. For example, if the counter value is set to 3, then the stimulation protocol 200 may refrains from applying a voltage to any electrode until the counter value reaches 3. In other words, the stimulation protocol 200 may be configured to apply a stimulation to the CSN 114 every third detected QRS complex.

At a second decision 206, the stimulation protocol 200 may determine whether the counter value is equal to a configured threshold counter value (e.g., N). If no, the stimulation protocol 200 may refrain from stimulating any electrode. If yes, the stimulation protocol 200 proceeds to a second process 208, wherein the IPG 104 may apply a voltage (e.g., via the stimulation module 138) to one or more electrodes after a configurable X ms delay (e.g., 50, 100, 200, 300, or 400 ms, or a delay within a range defined by any pair of these durations). Thus, if the threshold counter condition is met, then the IPG 104 may apply the voltage to one or more electrodes after the delay. The duration may begin upon detection of the QRS event, and end once X ms is reached, upon which the one or more electrodes are energized. Stimulation may be applied for a duration of, e.g., 200, 250, 300, 350, or 400 ms, or for a duration within a range defined by any pair of these durations). It should be noted that the human subject with the IPG 104 or a medical practitioner may use an external device 130 to set (e.g., via the application module 140) X ms delay value. Moreover, although the unit of time in this example is given in “ms,” the IPG 104 may be configured using any suitable temporal indicator (e.g., processor clock cycles, microseconds, nanoseconds, etc.).

At a third process 210, the stimulation protocol 200 may reset the counter after stimulation of the electrode(s). Once the counter is reset, the IPG 104 may proceed to monitor for another QRS event.

### Examples of Stimulation Protocol Adjustments Triggered by Physiological Events

Aspects of the disclosure are directed to stimulation protocols that are adjusted by detected physiological changes in the human subject with the IPG 104 device. As discussed, limiting the stimulation to less than every QRS complex increases the power efficiency of the IPG 104 by improving battery 142 life, while also eliminating or reducing undesirable side effects of the stimulation. However, stimulation can also be limited in other scenarios to further improve battery life and reduce undesirable side effects.

For example, a particular human subject may experience high blood pressure during the day, while also experiencing relatively lower blood pressure at night during sleep. In this example, the IPG 104 may refrain from performing any stimulation while the subject is sleeping. Similarly, the subject may experience a higher blood pressure during exercise or activity, during periods of a relatively lower respiration rate, etc., or simply a lower blood pressure without an identifiable cause. In any of these examples, the IPG 104 may detect such a physiological event (e.g., higher heart rate and/or respiration rate indicating the subject is exercising, lower respiration rate or other physiological marker indicating the subject is sleeping, or simply a lower blood pressure indicating that stimulation of the subject's CSN 114 is unnecessary) and change or adjust the stimulation protocol to reduce or increase CSN 114 stimulation.

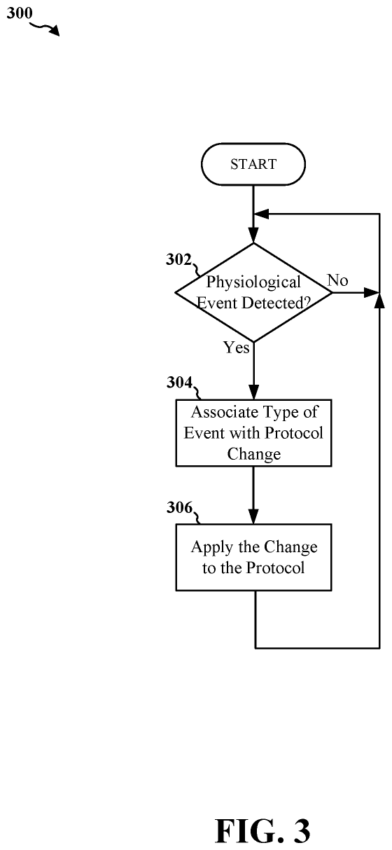

FIG. 3 is a flow chart illustrating an example process 300 for adjusting a stimulation protocol configured to stimulate the CSN 114. It should be noted that one or more aspects of the illustrated process 300 may be performed in parallel with the stimulation protocol 200 illustrated in FIG. 2. For example, the IPG 104 may refrain from counting a QRS complex if the subject is determined to be sleeping, exercising, or is otherwise experiencing relatively lower blood pressure. However, upon any one or more of these events no longer being true, the IPG 104 may resume counting each detected QRS complex and performing the process illustrated in FIG. 2.

At a first decision 302, the IPG 104 may detect (e.g., via the detecting module 134) a physiological event associated with the subject. Such events may be configurable by the subject or a medical practitioner via an external device 130 and the application module 140. The IPG 104 may be configured to detect whether the subject is sleeping, exercising, etc., based on any suitable physiological markers. In one example, the IPG 104 may be configured to detect that the subject is sleeping based on a configurable time of day, heart rate, and/or another suitable marker. For example, a controller 132 of the IPG 104 may determine that the subject is asleep based on sensor data from an internal measurement unit (IMU) indicating that the subject has been stationary, or engaged in little movement, over a predetermined time period. In some aspects, sensor data from a heart rate sensor and/or an IMU may be used to detect or determine respiration or a respiration cycle, and this parameter may be used to determine whether the subject is awake asleep. In some aspects, the subject may indicate that they intend to sleep using an interface provided by an application or mobile device communicatively linked to the controller 132 of the IPG 104, optionally with a built in delay set to turn off or reduce stimulation. Here, upon reaching a configured time of day and/or another threshold associated with another marker (e.g., detected by an external sensor), the IPG 104 may determine that a physiological event has occurred. In another example, the IPG 104 may be configured to detect that the subject's blood pressure has dropped to be equal to or less than a threshold value, for a threshold duration of time. For example, the subject's systolic blood pressure may be 130 mmHg or 120 mmHg. The IPG 104 may be configured to detect the occurrence of an instantaneous change (e.g., greater than a minimum threshold), or to detect that the subject's blood pressure is below a minimum threshold for a predetermined period of time (e.g., 10, 20, 30, 60, 120 minutes). With both threshold conditions met, e.g., the IPG 104 may determine that a physiological event has occurred. It should be noted that any threshold condition indicative of whether a physiological event has occurred may be configurable by a medical practitioner or the subject.

If a physiological event has occurred, the process 300 may proceed to a first sub-process 304 wherein the IPG 104 may associate the detected event to a particular change to the stimulation protocol 200. For example, if the IPG 104 detected that the subject is sleeping, it may change the stimulation protocol 200 such that the IPG 104 (e.g., the stimulation module 138) refrains from applying voltage to any of the electrodes for a configurable period of time. For example, a controller 132 of the IPG 104 may be configured to refrain from applying voltage for a programmable time period (e.g., a mean sleep duration), or to check periodically for signs that the subject is no longer sleeping (e.g., motion, a heart rate, or a respiration rate above a predetermined threshold). In some aspects, a controller 132 of the IPG 104 may be configured to periodically check the subject's blood pressure via one or more sensors and to determine that sleep has ended when the blood pressure exceeds a predetermined threshold. Any of these parameters may be measured or determined, e.g., at least once every 1, 2, 3, 4, 5, 6, 7, 8, 9, 10, 20, 30, 40, 50, or 60 minutes. Upon expiration of the configurable period of time, or a determination that the subject is no longer sleeping, the stimulation protocol 200 may resume normal operation.

In another example, if the IPG 104 detects that the subject is exercising or has a relatively high activity level (e.g., as indicated by heart and/or respiratory rate being greater than or equal to a configurable threshold value(s)), the IPG 104 may change the stimulation protocol 200 such that the IPG 104 refrains from applying voltage to any of the electrodes until the heart and/or respiratory rate is less than or equal to the configurable threshold value(s). In some aspects, threshold values for heart rate may be a rate above 100, 120, 140, 160, 180 or 200 BPS. The threshold may be programmable and subject-specific. In some aspects, a heart rate threshold may be determined in relation to the subject's resting heart rate, e.g., an increase of 1.2×, 1.3×, 1.4×, etc. may be used to determine that the subject is exercising or has a relatively high activity level. For respiration rate, a threshold of 20, 25, 30, 40, or 50 breaths per minute, or 1.5×, 2×, 3×, or 4× increase compared to a subject's resting respiration rate, may be used in some aspects. Upon the heart and/or respiratory rate becoming less than or equal to the configurable threshold value(s), the stimulation protocol 200 may resume normal operation.

At a second sub-process 306, the IPG 104 may apply the change associated with the type of physiological event to the stimulation protocol 200. As discussed above, this may include refraining from further stimulation during a (pre-)defined duration of time (e.g., 30, 45, 60, 75 or 90 minutes) or until a threshold condition is met (e.g., heart rate, blood pressure, or respiration rate returning to a resting level, as determined using any of the thresholds used as a basis for detecting exercise). It should be noted that a detected physiological event may further change or cancel a previously made change to the stimulation protocol based on a previously detected physiological event. For example, the IPG 104 may refrain from stimulation for a period of time based on a detection that the subject is sleeping. However, if the IPG 104 detects that the subject's blood pressure meets or exceeds a configurable threshold value within the period of time that the IPG 104 has refrained from stimulation, the IPG 104 may resume the normal stimulation protocol 200.

In another example, the stimulation protocol 200 may be configured such that the IPG 104 only stimulates the electrodes after N detected QRS complexes (e.g., where N=5). However, at the first decision 302, the IPG 104 may detect that the subject blood pressure has increased to meet or exceed a configurable threshold value. In such an example, at the first sub-process 304, the IPG 104 may determine a protocol change associated with the increased blood pressure. For example, the protocol change may be to reduce N to a lower number than 5. At the second sub-process 306, the IPG 104 may then set N=3 for example, or whatever value is configured for a detected increase in blood pressure.

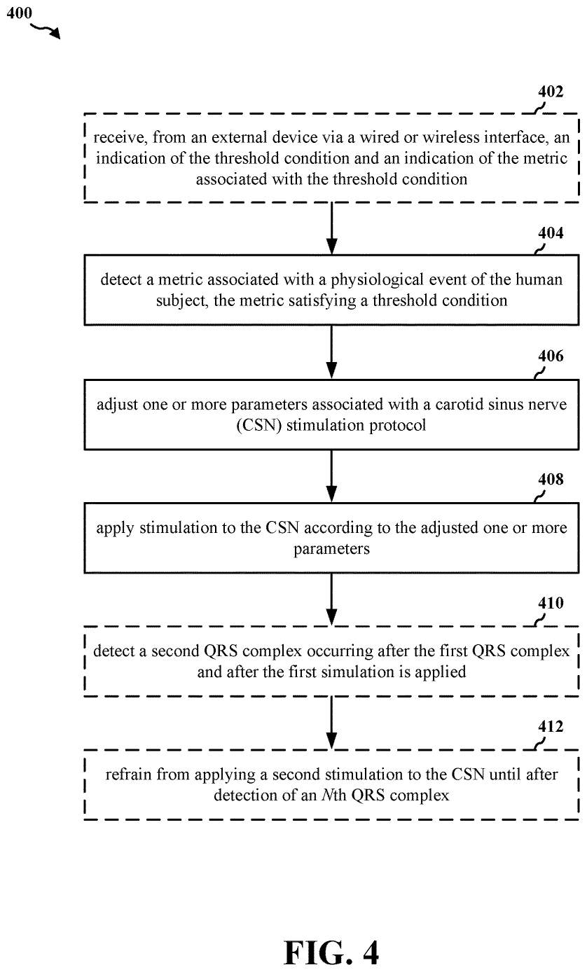

FIG. 4 is a flow diagram illustrating an example method 400 for reducing blood pressure in a human subject. The method may be performed by the IPG 104 and the arterial cuff 102 illustrated in FIG. 1. Specifically, the method may be performed by one or more memories, processors, electrodes (e.g., the one or more memories 144, the one or more processors/controllers 132, communication system 110, arterial cuff 102, electrodes 128, etc. of FIG. 1). Dashed lines indicate optional aspects of the illustrated method 400.

At 402, the IPG may receive, from an external device via a wired or wireless interface, an indication of the threshold condition and an indication of the metric associated with the threshold condition. For example, 402 may be performed by the communication system 110 of FIG. 1. Here, the human subject and/or medical practitioner may configure the IPG 104 via an external device 130. For example, the configuration may indicate particular values (e.g., an integer indicating millimeters of mercury (mmHg)) and an associated metric (e.g., blood pressure). For example, systolic blood pressure of 120 mmHG if therapy results in blood pressure control. However, therapy may also result in lowering blood pressure to a number that is above a controlled level, e.g., 160 mmHg (where the subject's baseline was 180). As such, in some aspects this threshold may be programmable to allow for subject-specific treatment design. In this example, the if the IPG 104 detects the subject's blood pressure meets, exceeds, or is less than the integer value, the IPG 104 may be triggered to perform or refrain from performing a particular action. Upper and lower thresholds may be used, as some cases therapy may lower blood pressure too much, in which case it would be desirable to reduce or eliminate stimulation.

At 404, the IPG may detect a metric associated with a physiological event of the human subject, the metric satisfying a threshold condition. For example, 404 may be performed by the detecting module 134 of FIG. 1. Here, the IPG may detect one or metrics having an associated measurement or value that satisfies a threshold condition. For example, the IPG may detect that the subject's blood pressure (e.g., metric associated with a physiological event) satisfies a threshold condition (e.g., measurement of the blood pressure is greater than, less than, or equal to a threshold value). In some aspects, the IPG may be configured to determine that the subject's blood pressure is maintained below 130 mmHg, or to determine that there has not been a change of >20 mmHg within a defined time period. In another example, the IPG may detect that it is currently a time of day (e.g., threshold condition) indicative of a time that the subject is sleeping (e.g., metric associated with a physiological event).

At 406, the IPG may adjust one or more parameters associated with a carotid sinus nerve (CSN) stimulation protocol. For example, 404 may be performed by the adjusting module 136 of FIG. 1. Here, in response to a triggering metric and condition, the IPG may adjust a stimulation protocol. For example, if the IPG detects that the subject is sleeping, it may refrain from stimulating the CSN for a configurable period of time. In another example, if the IPG detects that the subject's blood pressure has elevated to a level that satisfies a threshold condition, then it may increase the frequency, current, pulse width, or duty cycle at which stimulation is applied to the CSN (or lower the QRS interval, N).

At 408, the IPG may apply stimulation to the CSN according to the adjusted one or more parameters. For example, 408 may be performed by the stimulation module 138 of FIG. 1. Here, the IPG may apply or refrain from applying stimulation to the electrodes 128, based on the current stimulation protocol and/or any adjustments made to it.

At 410, the IPG may detect a second QRS complex occurring after the first QRS complex and after the first stimulation is applied. For example, 410 may be performed by the detecting module 134 of FIG. 1. Here, the IPG may be configured to stimulate in response to less than all detected QRS complexes. Thus, if the IPG stimulates the CSN in response to a first QRS complex, it may refrain from stimulating the CSN in response to a second QRS complex.

At 412, the IPG may refrain from applying a second stimulation to the CSN until after detection of an Nth QRS complex (e.g., after 1, 2, 3, 4, 5, 6, 7, 8, 9, or 10 QRS complexes). For example, 412 may be performed by the stimulation module 138 of FIG. 1. Here, the stimulation module may refrain from stimulating the subject's CSN until after a counter reaches a threshold value (e.g., N).

In certain aspects, the metric comprises at least one of a heart rate, a respiration rate, an activity level, a time of day, or a blood pressure.

In certain aspects, the one or more parameters associated with the CSN stimulation protocol comprises: a periodicity or frequency associated with the application of stimulation, a timing associated with the application of stimulation, an intensity of the stimulation, or a duration of the application of stimulation.

In certain aspects, the physiological event is a first QRS complex, wherein the stimulation applied to the CSN is a first stimulation, and wherein first stimulation to the CSN is applied X milliseconds after detection of the first QRS complex.

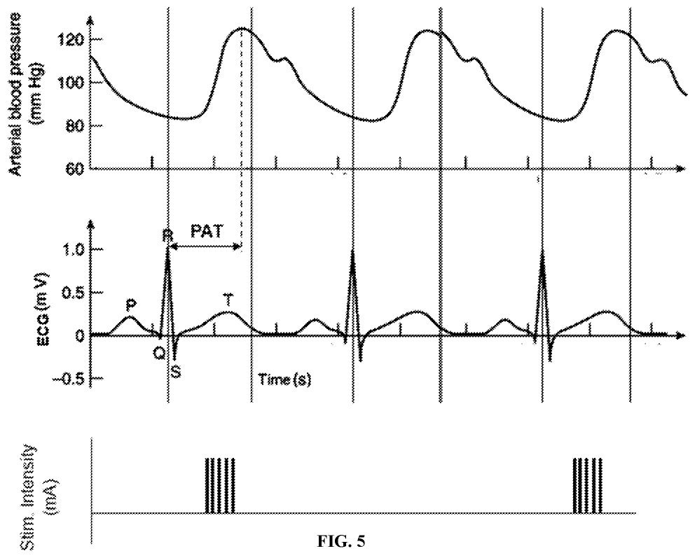

In certain aspects, stimulation to the CSN is applied X milliseconds after every Nth detected QRS complex. For example, FIG. 5 illustrates a hypothetical set of waveforms of arterial blood pressure, ECG, and stimulation amplitude, for a subject. As shown, a burst of stimulation is delivered during the pulse arrival time (PAT) every N cardiac cycle(s), wherein N is determined experimentally during a fitting procedure, based on the maximum number of cardiac cycles between stimulation events that results in an average systolic blood pressure reading within a desired range (e.g., 115-125 mmHg). In this example N=2. However, for other subjects, stimulation administered according to an alternative schedule may suffice (e.g., where N=3, 4, 5, etc.). Furthermore, the criteria for determining an acceptable outcome may also vary. In this case, a desired blood pressure range of 115-125 mmHg was targeted. In some aspects, a stimulation schedule may be found to be acceptable when it results in a desired blood pressure level (e.g., any integer value within the range of 115-125 mmHg, or any subrange defined by a pair of such integers), or when it crosses a threshold (e.g., dropping below 125 mmHG, or any other integer value within the range of 115-125 mmHg). The subject's blood pressure level may comprise an average value (e.g., measured over a period of 10, 20, 30, 40, 50, or 60 seconds, or within a subrange defined by any pair of these durations)

### Additional Considerations

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

### Example Aspects

The following examples are illustrative only and may be combined with aspects of other embodiments or teachings described herein, without limitation.

Example 1 is a method for reducing blood pressure in a human subject, comprising: detecting a metric associated with a physiological event of the human subject, the metric satisfying a threshold condition; adjusting one or more parameters associated with a carotid sinus nerve (CSN) stimulation protocol based on the detected metric; and applying stimulation to the CSN according to the adjusted one or more parameters.

Example 2 is the method of Example 1, wherein the metric comprises at least one of a heart rate, a respiration rate, an activity level, a time of day, or a blood pressure.

Example 3 is the method of any of Examples 1 and 2, further comprising: receiving, from an external device via a wired or wireless interface, an indication of the threshold condition and an indication of the metric associated with the threshold condition.

Example 4 is the method of any of Examples 1-3, wherein the one or more parameters associated with the CSN stimulation protocol comprises: a stimulation frequency or duty cycle associated with the application of stimulation, a timing associated with the application of stimulation, an intensity of the stimulation, or a duration of the application of stimulation.

Example 5 is the method of any of Examples 1-4, wherein the physiological event is a first QRS complex, wherein the stimulation applied to the CSN is a first stimulation, and wherein first stimulation to the CSN is applied X milliseconds after detection of the first QRS complex.

Example 6 is the method of Example 5, further comprising: detecting a second QRS complex occurring after the first QRS complex and after the first stimulation is applied; and refraining from applying a second stimulation to the CSN until after detection of an Nth QRS complex.

Example 7 is the method of any of Examples 1-6, wherein stimulation to the CSN is applied X milliseconds after every Nth detected QRS complex.

Example 8 is an implantable apparatus configured for reducing blood pressure in a human subject, comprising means for performing a method in accordance with any one of examples 1-7.

Example 9 is a non-transitory computer-readable medium comprising instructions that, when executed by an implantable apparatus, cause the implantable apparatus to perform a method in accordance with any one of examples 1-7.

Example 10 is an implantable apparatus, comprising: one or more memories, individually or in combination, having instructions; and one or more processors, individually or in combination, configured to execute the instructions to cause the implantable apparatus to perform a method in accordance with any one of examples 1-7.

What is claimed is:

1. An implantable apparatus configured for reducing blood pressure in a human subject, comprising:
one or more memories, individually or in combination, having instructions; and
one or more processors, individually or in combination, configured to execute the instructions to cause the apparatus to:
detect a metric associated with a physiological event of the human subject, the metric satisfying a threshold condition;
adjust one or more parameters associated with a carotid sinus nerve (CSN) stimulation protocol based on the detected metric; and
apply stimulation to the CSN according to the adjusted one or more parameters.

2. The apparatus of claim 1, wherein the metric comprises at least one of a heart rate, a respiration rate, an activity level, a time of day, or a blood pressure.

3. The apparatus of claim 1, wherein the one or more processors, individually or in combination, are further configured to:
receive, from an external device via a wired or wireless interface, an indication of the threshold condition and an indication of the metric associated with the threshold condition.

4. The apparatus of claim 1, wherein the one or more parameters associated with the CSN stimulation protocol comprises: a periodicity or frequency associated with the application of stimulation, a timing associated with the application of stimulation, an intensity of the stimulation, or a duration of the application of stimulation.

5. The apparatus of claim 1, wherein the physiological event is a first QRS complex, wherein the stimulation applied to the CSN is a first stimulation, and wherein first stimulation to the CSN is applied X milliseconds after detection of the first QRS complex.

6. The apparatus of claim 5, wherein the one or more processors, individually or in combination, are further configured to:
detect a second QRS complex occurring after the first QRS complex and after the first stimulation is applied; and
refrain from applying a second stimulation to the CSN until after detection of an Nth QRS complex.

7. The apparatus of claim 1, wherein stimulation to the CSN is applied X milliseconds after every Nth detected QRS complex.

8. A method for reducing blood pressure in a human subject, comprising:
detecting a metric associated with a physiological event of the human subject, the metric satisfying a threshold condition;
adjusting one or more parameters associated with a carotid sinus nerve (CSN) stimulation protocol; and
applying stimulation to the CSN according to the adjusted one or more parameters.

9. The method of claim 8, wherein the metric comprises at least one of a heart rate, a respiration rate, an activity level, a time of day, or a blood pressure.

10. The method of claim 8, further comprising:
receiving, from an external device via a wired or wireless interface, an indication of the threshold condition and an indication of the metric associated with the threshold condition.

11. The method of claim 8, wherein the one or more parameters associated with the CSN stimulation protocol comprises: a periodicity or frequency associated with the application of stimulation, a timing associated with the application of stimulation, an intensity of the stimulation, or a duration of the application of stimulation.

12. The method of claim 8, wherein the physiological event is a first QRS complex, wherein the stimulation applied to the CSN is a first stimulation, and wherein first stimulation to the CSN is applied X milliseconds after detection of the first QRS complex.

13. The method of claim 12, further comprising:
detecting a second QRS complex occurring after the first QRS complex and after the first stimulation is applied; and
refraining from applying a second stimulation to the CSN until after detection of an Nth QRS complex.

14. The method of claim 8, wherein stimulation to the CSN is applied X milliseconds after every Nth detected QRS complex.

15. A non-transitory, computer-readable medium comprising computer executable code, the code when executed by one or more processors causes the one or more processors to, individually or in combination, perform operations configured to reduce blood pressure in a human subject, the operations comprising:
detecting a metric associated with a physiological event of the human subject, the metric satisfying a threshold condition;
adjusting one or more parameters associated with a carotid sinus nerve (CSN) stimulation protocol; and
applying stimulation to the CSN according to the adjusted one or more parameters.

16. The non-transitory, computer-readable medium of claim 15, wherein the metric comprises at least one of a heart rate, a respiration rate, an activity level, a time of day, or a blood pressure.

17. The non-transitory, computer-readable medium of claim 15, wherein the operations further comprise:
receiving, from an external device via a wired or wireless interface, an indication of the threshold condition and an indication of the metric associated with the threshold condition.

18. The non-transitory, computer-readable medium of claim 15, wherein the one or more parameters associated with the CSN stimulation protocol comprises: a periodicity or frequency associated with the application of stimulation, a timing associated with the application of stimulation, an intensity of the stimulation, or a duration of the application of stimulation.

19. The non-transitory, computer-readable medium of claim 15, wherein the physiological event is a first QRS complex, wherein the stimulation applied to the CSN is a first stimulation, and wherein first stimulation to the CSN is applied X milliseconds after detection of the first QRS complex.

20. The non-transitory, computer-readable medium of claim 19, wherein the operations further comprise:
detecting a second QRS complex occurring after the first QRS complex and after the first stimulation is applied; and
refraining from applying a second stimulation to the CSN until after detection of an Nth QRS complex.
